(19) United States
(12) Patent Application Publication
(10) Pub. No.: US 2024/0130966 A1
(43) Pub. Date: Apr. 25, 2024

(54) INHALABLE IMATINIB FORMULATIONS, MANUFACTURE, AND USES THEREOF

(71) Applicant: Aerovate Therapeutics, Inc., Waltham, MA (US)

(72) Inventors: Ben Dake, Waltham, MA (US); Ralph Niven, San Francisco, CA (US)

(21) Appl. No.: 18/276,396

(22) PCT Filed: Feb. 15, 2022
(86) PCT No.: PCT/US2022/016422

§ 371 Date: Aug. 8, 2023

Related U.S. Application Data
(60) Provisional application No. 63/149,446, filed on Feb. 15, 2021.

(51) Int. Cl. A61K 9/00; A61K 9/14; A61K 31/506

(52) U.S. Cl. CPC A61K 9/0075; A61K 9/14; A61K 31/506

(57) ABSTRACT

The invention relates to inhalable imatinib formulations, manufacture, and uses thereof.

### CROSS REFERENCE TO RELATED APPLICATIONS

The present application claims the benefit of and priority to U.S. Provisional Patent Application No. 63/149,446, filed Feb. 15, 2021, the content of which is hereby incorporated by reference herein in its entirety.

### FIELD OF THE INVENTION

The invention relates to inhalable imatinib formulations, manufacture, and uses thereof.

### BACKGROUND

Pulmonary arterial hypertension (PAH) is a condition involving elevated blood pressure in the arteries of the lungs with unknown causes and is differentiated from systemic hypertension. PAH is a progressive disease where resistance to blood flow increases in the lungs causing damage to the lungs, the pulmonary vasculature and the heart that can eventually lead to death. While symptoms are treatable with vasodilators and other medications, there is no known disease modifying therapy or cure and advanced cases can eventually require lung transplants.

Imatinib, especially the mesylate salt thereof, is a tyrosine kinase inhibitor approved for use in treating certain types of cancer. Imatinib's potential to inhibit the tyrosine kinase platelet-derived growth factor receptor (PDGFR) which is highly upregulated in the pulmonary arteries in cases of PAH, led to interest in its use in treating PAH. See, Olschewski, H, 2015, Imatinib for Pulmonary Arterial Hypertension—Wonder Drug or Killer Drug? Respiration, 89:513-514, incorporated herein by reference. To that end, studies have been conducted to determine the potential of imatinib in treating PAH and patients have been found to respond favorably to said treatment. Unfortunately, an unacceptable amount of severe adverse events including subdural hematoma blunted enthusiasm for the drug. Frost, et al., 2015, Long-term safety and efficacy of imatinib in pulmonary arterial hypertension, J Heart Lung Transplant, 34(11):1366-75, incorporated herein by reference.

### SUMMARY

Compositions and methods of the invention address problems with imatinib-based PAH treatments through the use of specialized formulations and delivery mechanisms. Particularly, the invention provides inhalable dry powder formulations of crystalline imatinib and/or salts thereof that can be used to treat PAH and other conditions of the pulmonary cardiovascular system. In preparing dry powder formulations, moisture is usually avoided, however, the present invention identifies a surprising outcome after brief exposure of the dry powder to high humidity. The present invention recognizes that such exposure can actually significantly increase the fine particle dose content in a given volume (e.g., a capsule) as well as improve the aerodynamic profile of the particles for deep lung penetration.

In certain embodiments, milled powder formulations may be exposed to humidity at levels of about 50%, 60%, 70%, 80%, 90% or more relative humidity (at room temperature) for a period of 30 minutes, 1 hour, 1.5 hours, 2 hours, 2.5 hours, 3 hours, 4, hours, 6 hours, 12 hours, 24 hours, or less. In preferred embodiments, dry powder formulations of the invention may be exposed to 80%-90% relative humidity for a period of about 3 hours. In certain embodiments the humidity exposure may result in an increase of about 10-20% in fine particle dose of a capsule fill dose and may provide for smaller aerodynamic diameters, exhibiting improved powder dispersion and deaggregation from a dry powder inhaler device using fixed operating conditions. Exposure may occur at any point after the micronization process including, after filling capsules with the formulation for use in dry powder inhalers. Moisture content may be a result of intentional exposure to higher relative humidity after micronization as discussed above.

In that manner, the invention provides inhalable formulations of imatinib and salts thereof that offer greater lung exposure than equivalent doses of imatinib or imatinib mesylate administered through conventional oral or IV routes as well as inhalable formulations no subjected to humidity exposure. Accordingly, a relatively high oral dose of imatinib or imatinib mesylate would be required to achieve the same target lung exposure as achieved by inhalation of the inventive formulations. Therefore, the use of inhalable formulations of the invention allows for therapeutic amounts of imatinib to reach the lungs for treatment of PAH and other conditions of the pulmonary cardiovascular system without the adverse events experienced with prolonged oral administration of imatinib mesylate.

In certain embodiments, compounds and methods of the invention provide imatinib or a salt thereof in an inhalable form having entirely or almost entirely a single crystal form (e.g., greater than 80%, 85%, 90%, 95%, 99% or 100% of a single crystal form), thereby allowing for controlled and predictable dosing and patient response. In certain embodiments, greater than 95% of imatinib or a salt thereof in the inhalable formulation may be present in a single crystal form.

In certain embodiments inhalable imatinib compounds may be micronized using dry milling to achieve the desired particle size for dry powder formulations for inhalation. Imatinib or appropriate salts thereof may be micronized to particle sizes of about 0.5 μm to about 5 μm mass median aerodynamic diameter (MMAD) for desired deep lung penetration. Inhaled products can be limited in terms of mass of powder that can be administered and certain imatinib salts will contribute significantly to the molecular weight of the inhaled compound. Accordingly, in certain embodiments, the imatinib free base may be preferred for efficient delivery of the active moiety to lung tissue. If required, various excipients or carriers can be added to imatinib or salts thereof before or after micronization depending on application. For example, carriers, excipients, conditioners, and force control agents may be included with lactose (which may be conditioned with various solvents to increase separation of imatinib during inhalation), magnesium stearate, leucine, isoleucine, dileucine, trileucine, lecithin, distearylphosphatidylcholine (DSPC) or other lipid-based carriers, or various hydrophilic polymers. The skilled artisan will appreciate that excipients or carriers are optional and that many embodiments of the invention do not require excipients or carriers.

Another advantage of the compounds and methods of the invention is the ability to exclude all or most amorphous imatinib from the formulation, even after micronization. As noted above, because crystal form can be important to drug pharmacokinetics and dosing, as well as physicochemical stability and avoiding amorphous content can therefore be important to providing predictable and efficient therapy. A small amount of amorphous content (e.g., 0.1 to 0.5% w/w) may be generated in milled powders. Without wishing to be bound by any particular scientific theory, it is believed that the aforementioned humidity exposure catalyzes transition of this small amount of newly generated amorphous content to the more stable crystalline state.

Because the inhalable formulations described herein can modulate the uptake of imatinib in the target tissue of the lungs or microvasculature, formulations of the invention can be used to treat various conditions of the pulmonary cardiovascular system while avoiding the adverse events associated with higher doses that are administered by other routes of administration that introduce the drug systemically prior to reaching the target tissue. For example, compounds and methods of the invention can be used to treat PAH as well as lung transplant rejection, pulmonary veno-occlusive disease (PVOD) and pulmonary hypertension secondary to other diseases like heart failure with preserved ejection fraction (HFpEF) or schistosomiasis. Dose ranges can include between about 10 mg to about 100 mg per dose for inhalation on a twice to four times per day schedule. About 0.1 mg to about 20 mg of the active imatinib compound may then be present within the lungs after inhalation.

In certain embodiments, formulations of the invention can include processing and administration of imatinib in free base form. Free base imatinib formulations of the invention can retain crystallinity after micronization and are less hygroscopic than certain other imatinib salts. The differential hygroscopicity may contribute to the selective adsorption of water by the small amounts of amorphous content which can then be shed after transition to the preferred crystalline form(s).

The inhalable formulation may be in a dry powder. In some embodiments, the inhalable formulation may be a suspension of crystalline imatinib. The imatinib may be present in a therapeutically effective amount to treat a condition of the pulmonary cardiovascular system, such as pulmonary arterial hypertension (PAH). The salt may be at least one selected from the group consisting of glycollate, isethionate, xinafoate, furoate, trifenatate, HCl, sulfate, phosphate, lactate, maleate, malate, fumarate, tartrate, succinate, adipate, citrate, and malonate. In preferred embodiments, the salt may be glycolate, malate, tartrate, malonate, isethionate, or citrate. The inhalable formulation may further include one or more carrier agents.

### BRIEF DESCRIPTION OF THE DRAWINGS

FIG. 1 illustrates a new generation impactor size distribution profile for pre and post exposure to humidity.

FIG. 2 illustrates a vapor sorption isotherm for samples of unmilled vs. milled powder.

FIG. 3 illustrates differential scanning calorimetry results for spray-dried imatinib.

### DETAILED DESCRIPTION

The invention relates to inhalable formulations of imatinib and salts thereof. Imatinib, as used throughout the application, refers to the free base compound unless a salt thereof is recited. Imatinib as the free base has the below structure.

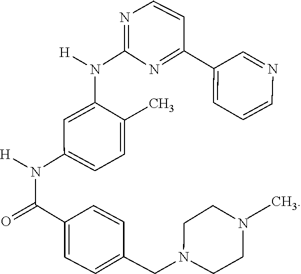

The methods and compositions described herein provide greater concentrations of imatinib in target lung tissue than obtained with equivalent doses administered orally or through IV. Accordingly, methods and compositions of the invention allow for treatment of conditions of the pulmonary cardiovascular system (e.g., PAH) with lower doses than would be required in systemic administration, thereby lowering the risk of adverse events including subdural hematoma (See, Frost et al.). Thus, the invention provides viable treatment methods for life threatening disease that were heretofore too risky for practical application.

In certain embodiments, compounds of the invention include formulations of imatinib or salts thereof. In preferred embodiments, the free base imatinib is used in a formulation (either in dry powder or suspension) for inhalation to treat a condition of the pulmonary cardiovascular system such as PAH. While free base imatinib is preferred due to its desirable characteristics when used in inhalable formulations, certain salt forms are also contemplated. In various embodiments, imatinib salts that were found to exhibit suitable thermal stability and few or single polymorphic forms include glycollate, isethionate, malonate, tartrate, and malate. Other salt forms contemplated herein are xinafoate, furoate, trifenatate, HCl, sulfate, phosphate, lactate, maleate, fumarate, succinate, adipate, and citrate

In various embodiments, micronized imatinib and salts thereof may be exposed to relative humidity of at least about 50, 60, 70, 80, or 90% at room temperature for a period of less than about 0.5, 1, 1.5, 2, 2.5, 3, 4, 5, 6, 12, 24 hours or less. In preferred embodiments, dry powder formulations of the invention may be exposed to 80%-90% relative humidity for a period of about 3 hours. In certain embodiments the humidity exposure may result in an increase of about 10-20% in fine particle composition in a capsule fill dose and may provide for smaller aerodynamic diameters.

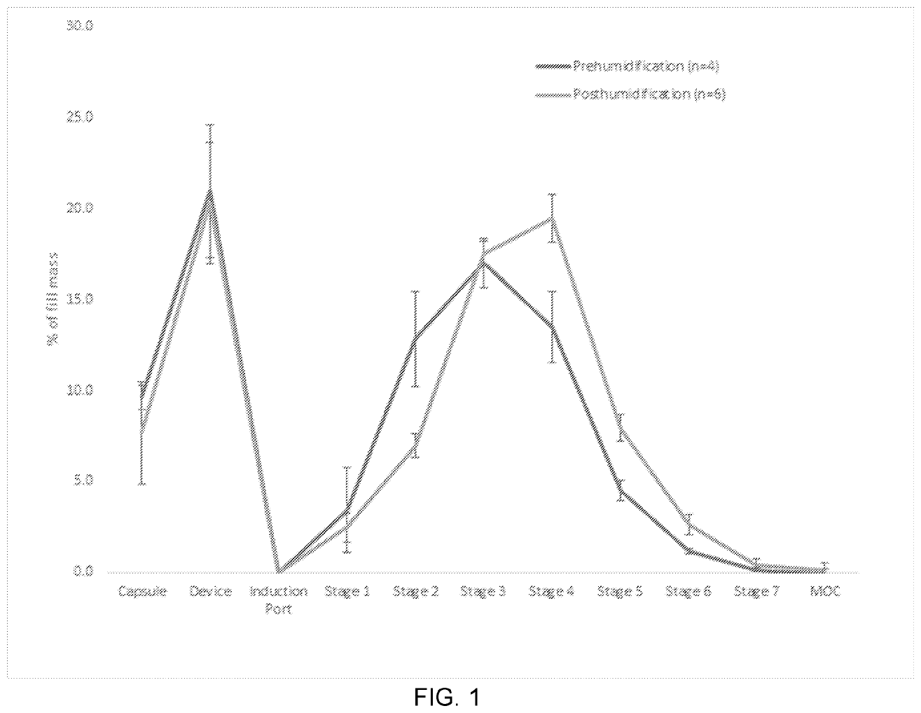

FIG. 1 illustrates some benefits of inhalable dry powder formulations of the invention prepared with exposure to humidity. Generator impactor profiles were prepared for ceramic milled powders both before and after conditioning with high humidity for a short time (80-90% RH at room temperature for ˜3 hours). Fine particle dose (i.e., expected lung dose represented by stage 3-MOC levels) is increased by 10-20% of the capsule fill dose. Furthermore, aerodynamic diameters as measured were shifted to smaller sizes preferred for lung penetration. In certain embodiments, compositions of the invention include dry powder formulations of imatinib or salts thereof comprising at least 1, 2, 3, 4, 5, 10, or 15% moisture content.

The advantages of humidity exposure are further illustrated in the pre- (N=7) and post-exposure (N=9) delivered doses as % of capsule fill detailed in TABLE 1. Again, the exposure consisted of 80%-90% relative humidity at room temperature for about 3 hours.

TABLE 1

| | Pre | Post |
|---|---|---|
| Delivered dose (% of fill) | 69.4 +/− 3.4 | 72.0 +/− 2.2 |
| FPF of fill (%) | 36.2 +/− 3.5 | 48.1 +/− 3.0 |
| FPF of emitted dose (%) | 52.3 +/− 6.3 | 66.8 +/− 4.3 |

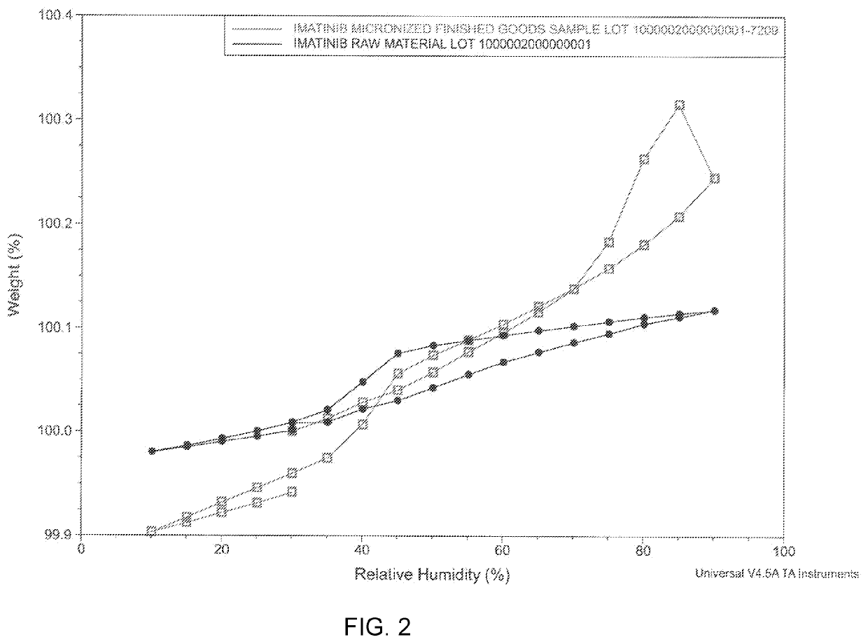

As shown in TABLE 1, the fine particle fraction (FPF) desired for lung penetration was increased in the fill volume, the delivered dose, and emitted dose after humidity conditioning. In general, higher apparent amorphous content in post-milled powders is correlated with poor aerodynamic particle size distribution. Without wishing to be tied to any particular scientific theory, it is believed that upon milling, small amounts of amorphous content can be generated in the powdered formulations. This is indicated by a loss in weight as the RH reaches the 80-90% range. FIG. 2 shows a vapor sorption isotherm for samples of unmilled vs. milled powder. A loss of weight at ˜85% RH shown in FIG. 2 suggests that a transition has taken place consistent with the amorphous content in the milled powder transforming to a crystalline condition. The uptake of moisture in the milled powder is higher than that of the unmilled powder which shows no transition and virtually no hysteresis. Furthermore, upon repeating the adsorption-desorption cycle in the milled powder no transition was apparent indicating that the event is not reversible. For both unmilled and milled material the uptake of moisture was very low.

It is believed that the adsorbed moisture on the amorphous content increased the weight until relative humidity reaches the aforementioned levels at which point the adsorbed water helps catalyze a transition in the small amounts of amorphous content to the more stable crystalline state. The water is then shed resulting in the measured weight loss as RH reaches the 80 to 90% range. Typically, amorphous content levels in the 0.1 to 0.5% w/w range are observed as a result of micronization but in some milled powders, in ‘over milling’ situations, or with any increased level of grinding on the powders, those amorphous content levels can be higher.

In certain embodiments a period of exposure to high humidity as described herein (85-90%) has been found to result in a change in the behavior of milled powders. For example, the post-humidified powders may be less likely to form plugs in a dosing gun relative to untreated powders. Furthermore, post-humidified powders were found to exhibit a substantial shift to smaller MMAD and higher fine particle fractions (e.g., <4.46 μm) at 60 liters per minute (LPM) in a next generation impactor (NGI) using a CDA Haler available from Emphasys Innovatec (Brazil) (e.g., 4 kPa drop at about 56 LPM).

Using dynamic vapor sorption analysis of a sample exposed to increasing increments of relative humidity at about 25° C., a loss in weight was noted at about 80% relative humidity. After desorption and cycle repetition, the loss in weight was not repeated. The loss in weight is believed to result from an amorphous to crystalline transition.

After exposing powders to humidity for a 12+ hour period and evaluating delivery performance before and after and then attempting to desorb powders at very low relative humidity (e.g., <5% RH) for a period of about 24 hours, no change in delivery performance was observed compared to the results of the post-humidification powders. It is therefore believed that the effects of humidity treatments (e.g., amorphous to crystalline transition) are not reversed upon removal from the humid conditions.

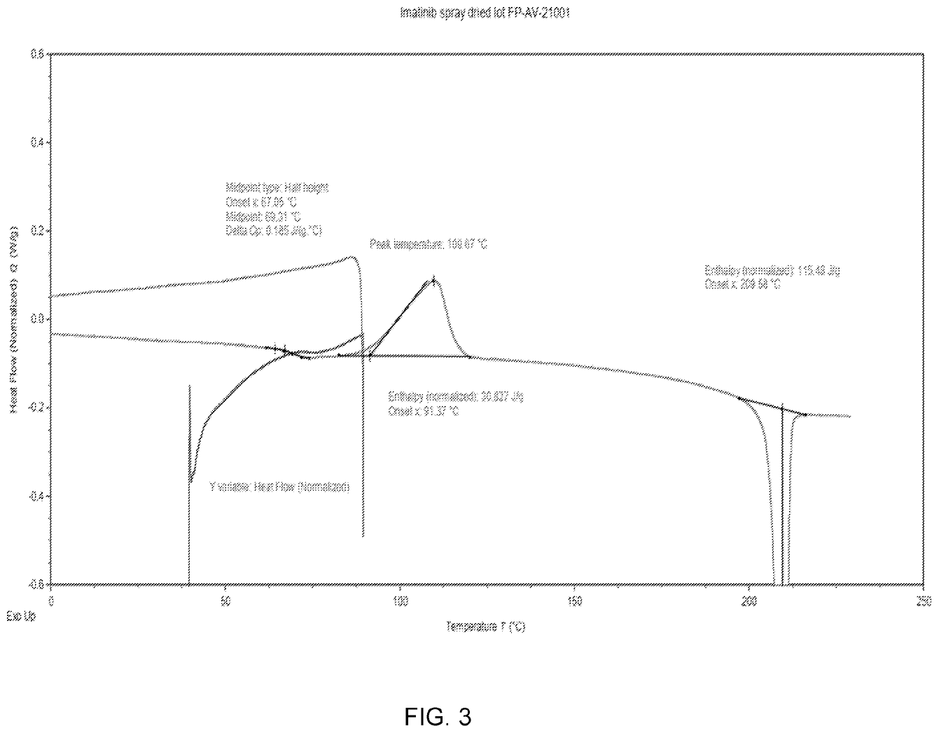

Without wishing to be bound to any particular theory, it is believed that scanning calorimetry studies will show that the glass transition temperature (T g) of amorphous imatinib decreases as a function of relative humidity, indicating that moisture may act as a catalyst (or lower the energy barrier) to facilitate the transition. The T_{g} under ambient conditions is believed to be about 67° C., based on an approximation of the T_{g} being ⅔ the melt temperature in Kelvin. In order to adequately detect the glass transition temperature (T g) of the amorphous content, a sample of imatinib was spray dried from an organic solution (ethanol) generating a high percentage of amorphous powder. This powder was subjected to differential scanning calorimetry, the results of which are shown in FIG. 3 and confirm a T_{g} of 67° C. and a notable exotherm with onset around 91° C. that may be associated with an amorphous to crystalline transition.

Thus, if exposure to humidity as described herein does reduce the T_{g}, it is believed, again without wishing to be bound to any particular theory, that the temperatures experienced during manufacture or handling could drive transition to crystalline form.

In certain embodiments, powders may be stored in hydroxypropyl methylcellulose (HPMC) or other capsules in bottles. Capsule moisture may be about 7% and substantially higher than that of the powder content therein. It may be that the equilibrium relative humidity within the capsule will accordingly be high. Accordingly, if powders have not undergone the aforementioned amorphous to crystalline transition before capsule filling, they may transition during storage. Such a transition may account for observed improvements in delivery performance over the first few months of storage in most circumstances. Accordingly, in certain embodiments, systems and methods of the invention may include storing inhalable formulations of the invention in a capsule at an increased relative humidity before administration to a patient in order to reduce amorphous content and improve inhalable performance. Storage times may be at least 1 day, at least 1 week, at least 1 month, at least 2 months, at least 6 months, or more in various embodiments.

Another unexpected result obtained with methods and formulations of the invention is that imatinib formulations of the invention are significantly less hygroscopic than conventional imatinib mesylate compounds. Accordingly, the imatinib formulations of the invention are better suited for dry powder inhalation and can comprise less than 5% water content, less than 4%, less than 3%, less than 2%, or, in preferred embodiments, less than 1% water content.

Formulations and methods of the invention may be used in combination with those described in U.S. App. Pub. Nos. 2020/0360376, 2020/0360275, 2020/0360377, 2020/0360276, 2020/0360277, 2020/0375895, 2020/0360279, and 2020/0360477, the content of each of which is incorporated herein by reference.

In various embodiments, the imatinib formulations of the invention may be pharmaceutical compositions for use in treating various conditions of the pulmonary cardiovascular system, such as PAH. For example, imatinib is a potent inhibitor of the platelet-derived growth factor receptor (PDGFR). Accordingly, the compositions of the invention may be used to treat any disease or disorder that involves inhibition of PDGFR or other kinases sensitive to imatinib.

In certain embodiments, the compositions of the invention may be used to treat PAH. For treatment of PAH or other disorders, a therapeutically effective amount of a pharmaceutical composition of imatinib according to the various embodiments described herein can be delivered, via inhalation (e.g., via dry powder inhaler or nebulizer) to deliver the desired amount of imatinib compound to the target lung tissue.

Dosages for treating PAH and other conditions of the pulmonary cardiovascular system may be in the range of between about 10 mg to about 100 mg per dose for inhalation on once, twice or three times per day schedule. About 0.1 mg to about 20 mg of the active imatinib compound may then be present at the lung after inhalation. In certain embodiments about 10 mg to 30 mg of imatinib may be given in a capsule for a single dry-powder inhalation dose with about 5 mg to about 10 mg of the compound to be expected to reach the lungs. In inhalable suspension embodiments, imatinib may be present at about 0.3 to about 1 mg/kg in a dose and may be administered one to four times a day to obtain the desired therapeutic results.

In certain embodiments, imatinib formulations of the invention may be used to treat pulmonary hypertension as a result of schistosomiasis. See, for example, Li, et al., 2019, The ABL kinase inhibitor imatinib causes phenotypic changes and lethality in adult Schistosoma japonicum, Parasitol Res., 118(3):881-890; Graham, et al., 2010, Schistosomiasis-associated pulmonary hypertension: pulmonary vascular disease: the global perspective, Chest, 137(6 Suppl):20S-29S, the content of each of which is incorporated herein by reference.

Imatinib pharmaceutical compositions of the invention may be used to treat lung transplant recipients to prevent organ rejection. See, Keil, et al., 2019, Synergism of imatinib, vatalanib and everolimus in the prevention of chronic lung allograft rejection after lung transplantation (LTx) in rats, Histol Histopathol, 1:18088, incorporated herein by reference.

In certain embodiments, pharmaceutical compositions described herein can be used to treat pulmonary veno-occlusive disease (PVOD). See Sato, et al., 2019, Beneficial Effects of Imatinib in a Patient with Suspected Pulmonary Veno-Occlusive Disease, Tohoku J Exp Med. 2019 February; 247(2):69-73, incorporated herein by reference.

For treatment of any conditions of the pulmonary cardiovascular system for which imatinib may produce a therapeutic effect, compounds and methods of the invention may be used to provide greater concentration at the target lung tissue through inhalation along with consistent, predictable pharmacokinetics afforded by low polymorphism and amorphous content. The efficient localization of therapeutic compound at the target tissue allows for lower systemic exposure and avoidance of the adverse events associated with prolonged oral administration of imatinib mesylate.

When the compounds of the present invention are administered as pharmaceuticals, to humans and mammals, they can be given per se or as a pharmaceutical composition containing, for example, 0.1 to 99.5% (more preferably, 0.5 to 90%) of active ingredient, i.e., at least one a therapeutic compound of the invention and/or derivative thereof, in combination with a pharmaceutically acceptable carrier.

The effective dosage of each agent can readily be determined by the skilled person, having regard to typical factors each as the age, weight, sex and clinical history of the patient. In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The pharmaceutical compositions of the invention include a “therapeutically effective amount” or a “prophylactically effective amount” of one or more of the compounds of the present invention, or functional derivatives thereof. An “effective amount” is the amount as defined herein in the definition section and refers to an amount effective, at dosages and for periods of time necessary, to achieve the desired therapeutic result, e.g., a diminishment or prevention of effects associated with PAH. A therapeutically effective amount of a compound of the present invention or functional derivatives thereof may vary according to factors such as the disease state, age, sex, and weight of the subject, and the ability of the therapeutic compound to elicit a desired response in the subject. A therapeutically effective amount is also one in which any toxic or detrimental effects of the therapeutic agent are outweighed by the therapeutically beneficial effects.

A “prophylactically effective amount” refers to an amount effective, at dosages and for periods of time necessary, to achieve the desired prophylactic result. Typically, since a prophylactic dose is used in subjects prior to, or at an earlier stage of disease, the prophylactically effective amount may be less than the therapeutically effective amount. A prophylactically or therapeutically effective amount is also one in which any toxic or detrimental effects of the compound are outweighed by the beneficial effects.

Dosage regimens may be adjusted to provide the optimum desired response (e.g. a therapeutic or prophylactic response). For example, a single inhalable bolus may be administered, several divided doses may be administered over time or the dose may be proportionally reduced or increased as indicated by the exigency of the therapeutic situation. Actual dosage levels of the active ingredients in the pharmaceutical compositions of this invention may be varied so as to obtain an amount of the active ingredient which is effective to achieve the desired therapeutic response for a particular subject, composition, and mode of administration, without being toxic to the patient.

The term “dosage unit” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the compound, and (b) the limitations inherent in the art of compounding such an active compound for the treatment of sensitivity in individuals.

In some embodiments, therapeutically effective amount can be estimated initially either in cell culture assays or in animal models, usually mice, rabbits, dogs, or pigs. The animal model is also used to achieve a desirable concentration range and route of administration. Such information can then be used to determine useful doses and routes for administration in other subjects. Generally, the therapeutically effective amount is sufficient to reduce PAH symptoms in a subject. In some embodiments, the therapeutically effective amount is sufficient to eliminate PAH symptoms in a subject.

Dosages for a particular patient can be determined by one of ordinary skill in the art using conventional considerations, (e.g. by means of an appropriate, conventional pharmacological protocol). A physician may, for example, prescribe a relatively low dose at first, subsequently increasing the dose until an appropriate response is obtained. The dose administered to a patient is sufficient to effect a beneficial therapeutic response in the patient over time, or, e.g., to reduce symptoms, or other appropriate activity, depending on the application. The dose is determined by the efficacy of the particular formulation, and the activity, stability, or half-life of the compounds of the invention or functional derivatives thereof, and the condition of the patient, as well as the body weight or surface area of the patient to be treated. The size of the dose is also determined by the existence, nature, and extent of any adverse side-effects that accompany the administration of a particular vector, formulation, or the like in a particular subject. Therapeutic compositions comprising one or more compounds of the invention or functional derivatives thereof are optionally tested in one or more appropriate in vitro and/or in vivo animal models of disease, such as models of PAH, to confirm efficacy, tissue metabolism, and to estimate dosages, according to methods well known in the art. In particular, dosages can be initially determined by activity, stability or other suitable measures of treatment vs. non-treatment (e.g., comparison of treated vs. untreated cells or animal models), in a relevant assay. Formulations are administered at a rate determined by the LD50 of the relevant formulation, and/or observation of any side-effects of compounds of the invention or functional derivatives thereof at various concentrations, e.g., as applied to the mass and overall health of the patient. Administration can be accomplished via single or divided doses.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of humans and lower animals without undue toxicity, irritation, allergic response, and the like, and are commensurate with a reasonable benefit/risk ratio.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of preparing an inhalable formulation comprising:
micronizing imatinib particles; and
exposing the micronized imatinib particles to moisture.

2. The method of claim 1 wherein the micronized particles are exposed to about 50% or more relative humidity at room temperature.

3. The method of claim 1 wherein the micronized particles are exposed to about 75% or more relative humidity at room temperature.

4. The method of claim 1 wherein the micronized particles are exposed to about 80% or more relative humidity at room temperature.

5. The method of claim 4 wherein the micronized particles are exposed to between about 80% and about 90% relative humidity at room temperature.

6. The method of claim 1 wherein the exposure to moisture occurs for about 1 hour or less.

7. The method of claim 1 wherein the exposure to moisture occurs for about 2 hours or less.

8. The method of claim 1 wherein the exposure to moisture occurs for about 3 hours or less.

9. The method of claim 1 wherein the exposure to moisture occurs for about 3 hours or more.

10. The method of claim 1 wherein the exposing step occurs after micronization.

11. The method of claim 10 further comprising filling a capsule with the micronized imatinib particles.

12. The method of claim 11 wherein the exposing step occurs after filling.

13. The method of claim 1 wherein the inhalable formulation comprises a higher ratio of fine particle dose relative to an equivalent inhalable formulation not subjected to the exposure step.

14. An inhalable formulation comprising imatinib or a salt thereof, comprising crystalline dry powder imatinib or a salt thereof and at least about 1% moisture content.

15. The inhalable formulation of claim 14 comprising imatinib or a salt thereof, comprising crystalline dry powder imatinib or a salt thereof and at least about 5% moisture content.

16. The inhalable formulation of claim 15 comprising imatinib or a salt thereof, comprising crystalline dry powder imatinib or a salt thereof and at least about 10% moisture content.

17. The inhalable formulation of claim 16 comprising imatinib or a salt thereof, comprising crystalline dry powder imatinib or a salt thereof and at least about 15% moisture content.
